(19) United States
(12) Patent Application Publication
(10) Pub. No.: US 2023/0233497 A1
(43) Pub. Date: Jul. 27, 2023

(54) COMPOSITION FOR POLYAMINE BIOSYNTHESIS

(71) Applicant: BM Health GmbH, Wien (AT)

(72) Inventor: Christian R. NOE, Wien (AT)

(73) Assignee: BM Health GmbH, Wien (AT)

(21) Appl. No.: 17/909,163

(22) PCT Filed: Mar. 3, 2021
(86) PCT No.: PCT/EP2021/055346

§ 371 Date: Sep. 2, 2022

(30) Foreign Application Priority Data
Mar. 4, 2020 (AT) A50162/2020

(51) Int. Cl. A61K 31/198; A23L 33/175; A61K 31/52; A61K 45/06

(52) U.S. Cl. CPC A61K 31/198; A23L 33/175; A61K 31/52; A61K 45/06; A61K 33/30

(57) ABSTRACT

The invention relates to a composition comprising at least one C4 component and at least one C3 component, wherein the C4 component consists of ornithine and/or arginine or a suitable derivative of ornithine and/or arginine, wherein the C3 component consists of methionine and/or S-adenosylmethionine or a suitable derivative of methionine and/or S-adenosylmethionine, and wherein the molar ratio between the C3 component and the C4 component is between 1:5 and 5:1. The invention further relates to a nutritional supplement.

### CROSS-REFERENCE TO RELATED APPLICATION(S)

This application is the National Stage of International Patent Application No. PCT/EP2021/055346, filed on Mar. 3, 2021, which claims priority from Austrian Patent Application No. A50162/2020 filed on Mar. 4, 2020, both of which are herein incorporated by reference in their entireties.

### BACKGROUND

The present invention relates to a composition for cell activation, in particular for induction of autophagy, and medicaments and nutritional supplements containing or consisting of said composition.

The phosphate group is an essential component of all life. DNA, just like RNA, is a biopolymer whose components are held together by diphosphate bridges. To activate a phosphated biomolecule, it must be transformed into a soluble salt. However, in order to dissolve a section of DNA or RNA as a sodium salt (or other alkali salt), large amounts of salt would be required, which would massively disturb the electrochemical potential of the cell. At the same time, the osmotic pressure would destroy the cell. It is only through the salt formation with spermine that a regulated function of DNA and RNA sections becomes possible at all. Therefore, a very special system is found in all cells, which may be called a “polyaminome”.

The polyaminome causes an enzyme apparatus to synthesise large quantities of the polyamines spermidine and spermine within a very short time when required, i.e. when cell activation occurs. Since these molecules have several basic amino groups (spermidine 3 and spermine 4), they do not generate any particular osmotic pressure during salt formation. Any action of the genome is thus usually preceded by the activation of the “polyaminome”, which is able to release the packed DNA from the histones. Although spermidine and spermine are much-described biomolecules and the presence of spermine as a counterion of DNA is known, the fundamental connection that the salt formation described here plays for the course of life processes (epigenetics) has so far been described at most rudimentarily in the scientific literature and its importance is hardly known.

Secondary messengers such as cAMP (cyclo-adenosine monophosphate) and cGMP (cyclo-guanosine monophosphate) are also diesters of phosphoric acid (diphosphates). The most important biological energy carrier ATP (adenosine triphosphate) is a monoester of phosphoric acid. Nucleotide diphosphates also play an important role in cellular events. Ultimately, the activity of proteins and sugar molecules is also regulated by phosphorylation and dephosphorylation by means of kinases. Since all the phosphates mentioned are polar molecules and are present in the cell as salts, they are usually tightly bound to basic sequences of proteins in the resting state. This is a basic requirement to avoid chaotic reactions in the cell.

ATP is the main energy carrier in biological systems. It would only form a full salt with 4 sodium ions, but only needs 1 molecule of spermine for this purpose. The two molecules spermidine and spermine are therefore fundamental drivers of metabolism.

In spermine, three carbon chains provided with amino groups are combined in the order 3-4-3, in spermidine there are two carbon chains with three and four methylene groups respectively. The biosynthesis of polyamines starts from the amino acids ornithine and methionine, which are decarboxylated respectively. First, ornithine is decarboxylated to the C4 diamine putrescine. Adenosine, in turn, first reacts with ATP to form S-adenosylmethionine to form an active S-activated intermediate, which is also decarboxylated. In the next step, an aminopropyl radical is transferred from the decarboxylated S-adenosylsmethionine to the ornithine. The resulting spermidine may further react with S-adenosylmethionine to spermine.

Ornithine is formed by urea splitting from arginine. In an alternative synthetic pathway, arginine is decarboxylated in the first step to the C4 component agmatine, from which putrescine is formed. The transfer of the aminopropyl radical may also occur directly to the agmatine. In each case, spermidine or spermine is formed by urea splitting from the guanidine structure.

Since the polyamines can basically activate all phosphate-containing compounds, they are also able to have an activating effect on the enzyme adenosine monophosphate kinase (AMPK), which, among other things, plays an important role in the transport through cellular membranes, as well as in the transport of glucose across the blood-brain barrier.

Spermidine has recently been marketed as an anti-ageing agent. Therefore, compositions containing spermidine are known in the prior art. The effect of spermidine is attributed to the process of autophagy.

### SUMMARY

In the context of the present invention, it is assumed that the phosphate-activating effect of the polyamine spermidine, which is directly supplied to the body, plays an important role in autophagy. Since the enzyme apparatus of the polyaminome, due to its fundamental importance, hardly allows for major gene defects of the various protein components, it can be assumed that a lack of spermidine or spermine is not due to defects of the enzyme apparatus, but primarily to a lack of the components needed for their synthesis.

To enable the organism to provide the required amounts of spermidine, it would be preferable to provide the starting materials necessary for the synthesis of the polyamines.

In view of the enzymatic synthesis apparatus that is universally present in the cells, a sufficient supply of the individual components of polyamine synthesis thus appears to be a particularly efficient way of providing spermidine and spermine.

The present invention is thus based on the task of creating a composition by means of which the respective required amount of polyamines, in particular of spermine and spermidine, can be made available to an organism.

These and further objects are solved by the subject matter of the present invention.

The invention thus relates to a composition comprising at least one C4 component and at least one C3 component, wherein the C4 component consists of ornithine and/or arginine or a suitable derivative of ornithine and/or arginine, and the C3 component consists of methionine and/or S-adenosylmethionine or a suitable derivative of methionine and/or S-adenosylmethionine.

In particular, in the context of the present invention, the term “C3 component” means that a (—CH₂—CH₂—CH₃) or a (—CH₂—CH₂—CH₂—) group is present in the molecular structure of this component. In particular, in the context of the present invention, the term “C4 component” means that a (—CH₂— CH₂—CH₂—CH₃) or a (—CH₂—CH₂—CH₂—CH₂—) group is present in the molecular structure of this component.

Suitable derivatives of the compounds mentioned are, for example, salts.

In a particularly preferred embodiment, the C3 component is S-adenosylmethionine and the C4 component is ornithine. These are the immediate precursor molecules of spermidine synthesis.

Optionally, it is provided that the molar ratio between the C3 component and the C4 component is between 1:5 and 5:1.

It is preferably provided that the molar ratio between the C3 component and the C4 component is from 0.8 to 1.2 parts of the C4 component and from 0.8 to 2.4 parts of the C3 component.

Optionally, it is provided that the C4 component and the C3 component form an amino acid component, the composition comprising between 80 wt.-% and 99 wt.-%, preferably between 80 wt.-% and 95 wt.-% of the amino acid component.

Optionally, it is provided that the composition does not contain any further amino acids in addition to the C4 component and the C3 component, or contains at most 5 wt.-%, preferably at most 2 wt.-%, of further amino acids. Preferably, no further amino acids are required for polyamine biosynthesis. However, smaller amounts of other amino acids may optionally be present.

Optionally, it is provided that the composition contains a carbohydrate, preferably glucose.

Optionally, it is provided that the composition additionally contains vitamins. In particular, the vitamin is at least one vitamin B. The vitamin B is preferably selected from one or more of: vitamin B6, vitamin B12, vitamin B9 (folic acid).

In the context of the present invention, vitamin B is in particular one or more vitamins from the group of B vitamins. A particularly preferred B vitamin is vitamin B6, which is a catalyst (coenzyme) for the decarboxylation reactions in biosynthesis. Vitamin B6 may be present in the composition in free form or as a salt, preferably in the form of pyridoxal-5-phosphate. Other B vitamins may also be present in free form or as a salt.

Optionally, it is provided that the vitamin B is contained in an amount between 0.05 wt.-% and 0.5 wt.-%, preferably between 0.05 wt.-% and 0.2 wt.-% relative to the total weight of the composition.

Optionally, it is provided that the composition contains a trace element, in particular a trace metal.

Optionally, it is provided that the trace metal is zinc.

Optionally, it is provided that the trace element is contained in an amount between 0.05 wt.-% and 0.2 wt.-% relative to the total weight of the composition.

Optionally, the composition according to the invention is intended for use in cell activation, in particular in the induction of cellular autophagy.

Another use of the composition according to the invention relates to the activation of transport across cellular membranes, in particular of glucose and other metabolites across the blood-brain barrier.

The invention further relates to medicaments containing or consisting of a composition according to the invention and/or nutritional supplements containing or consisting of a composition according to the invention.

Optionally, it is provided that the nutritional supplement contains between 70 wt.-% and 95 wt.-% of glucose. This allows for an optimised supply of glucose to the brain.

Optionally, it is provided that a nutritional supplement according to the invention additionally contains a transport mediator. Preferably, the transport mediator is an agent that activates the protein kinase (AMPK), i.e. an AMPK-activating agent. Most preferably, the transport mediator is an agent that has an activating effect on the glucose transporter GLUT1. Optionally, the transport mediator may be referred to as activator.

Phytopreparations may be used as transport mediators to increase the activity of the blood-brain barrier, especially those containing AMPK activators. Optionally, the phytopreparations may be prepared from the plant species Argemone mexicana, Berberis vulgaris, Coptis chinensis, Eschscholzia californica, Galega officinalis, Ginkgo biloba, Hydrastis Canadensis, Panax ginseng, Phellodendron amurense, Tinospora cordifolia or Xanthorhiza simplicissima

Preferred phytopreparations come from goat’s rue (Galega officinalis; containing galegin) and barberry (Berberis vulgaris). Optionally, also tea mixtures from TCM (Traditional Chinese Medicine) may be used. For example, hese include ginseng (Panax ginseng), gingko (Gingko biloba) and other tea blends that are thought to have a memory-enhancing or anti-dementia effect.

Alternatively, the transport mediator may be an estrogen-like natural substance or an extract of such a natural substance. Preferred examples of this may be resveratrol, genistein and curcumin; however, these examples are not intended to limit the possible transport mediators from the family of oestrogen-like natural substances. Any derivatives may optionally serve a comparable purpose as transport mediators.

Since omega-3 fatty acids unspecifically increase membrane fluidity, they may also be contained as transport mediator. In particular, an omega-3 fatty acid may be a fatty acid that is unsaturated at the omega-3 position. Fish oils containing omega-3 fatty acids are preferably used as emulsions, which may also improve the bioavailability of other water-insoluble transport mediators.

Optionally, a drug may also be used as a transport mediator. Preferred drugs according to the invention are biguanides, such as metformin, buformin or phenformin; however, the invention is not limited to this selection. Of course, other derivatives of these agents may also be effective as transport mediators.

Optionally, it is provided that the transport mediator is contained in in glucose-containing nutritional supplements in an amount between 0.5 wt.-% and 10 wt.-% relative to the amount of glucose.

The nutritional supplement according to the invention may also be used for cell activation, in particular for the induction of autophagy. Another use of the nutritional supplement according to the invention relates to the activation of the transport of glucose and other metabolites across the blood-brain barrier.

The nutritional supplement according to the invention may further be used for the prevention of mental performance loss. The nutritional supplement according to the invention may further be used for lowering the blood sugar level and as a dietary product against obesity.

Optionally, the nutritional supplement is free of amino acids other than those present in the composition according to the invention. The nutritional supplement according to the invention may optionally contain up to 5 wt.-%, preferably up to 2 wt.-%, more preferably 0 wt.-%, of amino acids, except one or more of ornithine, arginine, methionine and S-adenosylmethionine.

Optionally, a polyamine, in particular spermine and/or spermidine, may be included in a composition or nutritional supplement comprising one or more of the above additional components. In this case, the composition or nutritional supplement in particular does not contain any other C3 and C4 components.

Further features of the invention become apparent from the patent claims, the figures and the description of the preferably exemplary embodiments.

In the following, the present invention will be discussed in detail with reference to exemplary embodiments. The examples serve merely as illustrations to the invention and are not intended to limit the scope of protection of the claims.

### DETAILED DESCRIPTION OF THE PREFERRED EMBODIMENTS

### Example Group 1 With L-Methionine and L-Ornithine as Precursors

### Example 1a: Component Mixture

A base mixture of 110 mg L-methionine (about 65%) and 60 mg ornithine (about 35%) with 0.53 mg zinc bisglycinate and 0.28 mg vitamin B6 is prepared by mixing. The composition may be used in particular as an additive to medicinal products or as a premix for a nutritional supplement with other ingredients.

### Example 1b: Dextrose Drops

A component mixture according to example 1a is compressed with 2.44 g of dextrose with the addition of citric acid and flavourings of the types tropic, passion fruit and elderberry flower, using magnesium salts of fatty acids and hydrogenated vegetable oils as excipients, to form a tablet with a weight of 2.7 g.

Dextrose drops according to example 1b are, apart from the uses indicated in the description, particularly well suited as a means of remedying hypoglycaemic episodes in patients with type 1 diabetes (instead of conventional dextrose drops).

### Example 1c: Dextrose Drops With Activator

A tablet according to example 1b is compressed with an addition of 15 mg resveratrol. Dextrose drops according to example 1c are particularly suitable for supplying the brain with nutrients and thus for maintaining a normal brain metabolism.

### Example 1d: Sugar Sticks With Glucose

A powdered component mixture according to example 1a is mixed with 15 mg trans-resveratrol and 1.85 g dextrose and adjusted to a sweetening value corresponding to that of 4 g sucrose by adding small amounts of sweetener. The sweeteners used may be any authorised sweeteners, preferably 37.5 mg of a sodium cyclamate-sodium saccharin 10:1 mixture.

The nutritional supplement of example 1d may be used as a sweetener for tea, coffee and other beverages and is present in the form of pre-portioned sweetener sticks. In addition to the uses given in example 1d, it is suitable as a calorie-reduced sweetener for the treatment of obesity.

### Example 1e: Melissa Ginseng Instant Drink With Activator

To a component mixture according to example 1a, 15 mg of trans-resveratrol, 200 mg of melissa extract, 20 mg of ginseng extract, and 20 mg of peppermint extract are added. 4.4 g of dextrose and 76 mg of a sodium cyclamate-sodium saccharin 10:1 mixture are added and the mixture is packed into a can.

### Example 1f: Hop Ginseng Instant Drink With Activator

The preparation is analogous to that of example 1e. Instead of the melissa extract, 70 mg of hop extract (65-70% content), as well as 40 mg of orange flavouring and 2.5 mg of lemon flavouring are used.

The instant mixtures according to example 1e and 1f are dissolved in a beverage, preferably in an amount of 125 ml for a 5 g portion and preferably in water.

Instant blends with other flavour components may be produced analogously to examples 1e and 1f. The instant mixtures according to examples 1e and 1f are suitable, apart from the uses indicated in the description, as agents for reducing blood glucose levels in patients with prediabetes or type 2 diabetes. For this purpose, the instant mixtures may be sweetened with another sugar, a sugar substitute or sweetener as an alternative to dextrose, or they may be used unsweetened.

### Example Group 2 With S-Adenosylmethionine and L-Ornithine as Precursors

### Example 2a: Component Mixture

A base mixture of 160 mg S-adenosymethionine, 60 mg L-methionine and 60 mg ornithine with 0.28 mg vitamin B6 is prepared by mixing. The composition may be used in particular as an additive to medicinal products or as a premix for a nutritional supplement with other ingredients.

The composition may be used in particular as an additive to medicinal products or as a premix for a nutritional supplement with other ingredients.

### Example 2b: Dextrose Drops

A component mixture according to example 2a is compressed with 2.44 g of dextrose with the addition of citric acid and flavourings of the types tropic, passion fruit and elderberry flower, using magnesium salts of fatty acids and hydrogenated vegetable oils as excipients, to form a tablet with a weight of 2.7 g.

Dextrose drops according to examples 2a and 2b may also be produced with other flavours. They are, apart from the uses indicated in the description, particularly well suited as a means of remedying hypoglycaemic episodes in patients with type 1 diabetes (instead of conventional dextrose drops).

### Example 2c: Tablets With an Antidiabetic

A component mixture according to example 2a is compressed with 0.6 g of the antidiabetic drug metformin, 1.85 g of dextrose and 37.5 mg of a sodium cyclamate-sodium saccharin 10:1 mixture with the addition of citric acid and flavourings of the types tropic, passion fruit and elderberry blossom and using magnesium salts of fatty acids and hydrogenated vegetable oils as excipients to form a tablet with a weight of 2.7 g.

Tablets according to example 2c may be used to treat type 2 diabetes and to prevent neurodegeneration.

### Example 2d: Sugar Sticks With Glucose

A powdered component mixture according to example 2a is mixed with 15 mg trans-resveratrol and 1.85 g dextrose and by adding small amounts of sweeteners, any permitted sweeteners, preferably 37.5 mg of a sodium cyclamate-sodium saccharin 10:1 mixture may be used.

The nutritional supplement of example 2d may be used as a sweetener for tea, coffee and other beverages and is in the form of pre-portioned sweetener sticks. In addition to the uses given in example 1c, it is suitable as a calorie-reduced sweetener for the treatment of obesity.

### Example 2e: Flavoured Sugar Sticks With Glucose

Apple flavouring is added to a powdered component mixture according to example 2c.

The nutritional supplement of example 2e may preferably be used in 125 ml of water or soda water as a soft drink. As the product in example 2c, it is also suitable as a calorie-reduced sweetener for the treatment of obesity.

### Example 2f: Melissa Ginseng Instant Drink With Activator

To a component mixture according to example 2a, 15 mg of trans-resveratrol, 200 mg of melissa extract, 20 mg of ginseng extract and 20 mg of peppermint extract are added. 4.4 g of dextrose and 76 mg of a sodium cyclamate-sodium saccharin 10:1 mixture are added and the mixture is packed into a can.

### Example 2g: Coffee Ginseng Instant Drink With Activator

To a component mixture according to example 2a, 15 mg of trans-resveratrol and blonde roast coffee extract are added. 4.4 g of dextrose and 76 mg of a sodium cyclamate-sodium saccharin 10:1 mixture are added and the mixture is packed into a can. Instant blends with other flavour components may be produced analogously.

The instant mixtures according to examples 2f and 2 g are suitable, apart from the uses indicated in the description, as agents for reducing blood glucose levels in patients with prediabetes or type 2 diabetes. For this purpose, the instant mixtures may be sweetened with another sugar, a sugar substitute or sweetener as an alternative to dextrose, or they may be used unsweetened.

### Example Group 3: Arginine in Dual Function as Precursor and Endothelium-Relaxing Component

In this product group, arginine has a dual function both as an endothelium-relaxing agent (NO donor) and as a spermidine precursor (after decarboxylation via the pathway of agmatine with subsequent urea splitting to ornithine and to spermidine, respectively).

### Example 3a: Component Mixture

A base mixture of 140 mg L- methionine, 200 mg L-arginine hydrochloride and 0.28 mg vitamin B6 is prepared by mixing. The composition may be used in particular as an additive to medicinal products or as a premix for a nutritional supplement with other ingredients.

### Example 3b: Dextrose Drops

A component mixture according to example 3a is compressed with 40 micrograms of folic acid, 0.5 micrograms of vitamin B12 and 2.3 g of dextrose with the addition of citric acid and flavourings of the types tropic, passion fruit and elderberry flower, using magnesium salts of fatty acids and hydrogenated vegetable oils as excipients, to form a tablet with a weight of 2.7 g.

Dextrose drops according to example 3b may also be produced with other flavours. They have an effect against apoptotic processes. They are, apart from the uses indicated in the description, particularly well suited as a means of remedying hypoglycaemic episodes in patients with type 1 diabetes (instead of conventional dextrose drops).

### Example 3c: Sugar Sticks With Glucose

A powdered component mixture according to Example 3a is added with 40 micrograms of folic acid, 0.5 microgram of vitamin B12 and sweetened with 1.85 g of dextrose, and with 37.5 mg of a sodium cyclamate-sodium saccharin 10:1.

The nutritional supplement of example 2e may be used as a sweetener for tea, coffee and other beverages and is present in the form of pre-portioned sweetener sticks. In addition to the uses given in example 1c, it is effective against apoptotic processes and is suitable as a calorie-reduced sweetener for the treatment of obesity.

### Example 3d: Hop Ginseng Instant Drink With Activator

A component mixture according to example 3a is mixed with 40 micrograms of folic acid, 0.5 micrograms of vitamin B12, 50 mg of hop extract (65-70% content), 20 mg of ginseng extract, 20 mg of peppermint extract, and 40 mg of orange flavouring and 2.5 mg of lemon flavouring. 4.4 g of dextrose and 76 mg of a sodium cyclamate-sodium saccharin 10:1 mixture are added and the mixture is packed into a can.

The instant mixtures according to example 3e are dissolved in a beverage, preferably in an amount of 125 ml for a 5 g portion and preferably in water.

Instant blends with other flavour components may be produced analogously. Instant mixtures according to example 3e have an anti-apoptotic effect. They are suitable, apart from the uses indicated in the description, as agents for reducing blood glucose levels in patients with prediabetes or type 2 diabetes. For this purpose, the instant mixtures may be sweetened with another sugar, a sugar substitute or sweetener as an alternative to dextrose, or they may be used unsweetened.

The component mixtures of examples 1a, 2a and 3a are compositions according to the present invention and as such may be used as nutritional supplements without further additives. The above component mixtures achieve the effects according to the present invention.

The remaining examples are nutritional supplements within the meaning of the present invention, wherein further ingredients are contained, for example glucose, sweeteners and a transport mediator.

1-15. (canceled)

16. A composition comprising:
at least one C4 component consisting of ornithine and/or arginine or a suitable derivative of ornithine and/or arginine; and
and at least one C3 component consisting of methionine and/or S-adenosylmethionine or a suitable derivative of methionine and/or S-adenosylmethionine;
wherein a molar ratio between the least one C3 component and the least one C4 component is between 1:5 and 5:1.

17. The composition according to claim 16, wherein:
the molar ratio between the at least one C3 component and the at least one C4 component is from 0.8 to 1.2 parts of the C4 component and from 0.8 to 2.4 parts of the C3 component.

18. The composition according to claim 16, wherein:
the at least one C4 component and the least one C3 component form an amino acid component.

19. The composition according to claim 18, wherein:
the amino acid component is between 80 weight percent and 90 weight percent relative to total weight of the composition.

20. The composition according to claim 18, wherein:
the amino acid component is between 85 weight percent and 95 weight percent relative to total weight of the composition.

21. The composition according to claim 18, wherein:
the composition does not contain any further amino acid component in addition to the amino acid component formed from the at least one C4 component and the at least one C3 component.

22. The composition according to claim 18, wherein:
the composition contains at least one further amino acid component in addition to the amino acid component formed from the at least one C4 component and the at least one C3 component, wherein the at least one further amino acid component is at most 5 weight percent relative to total weight of the composition.

23. The composition according to claim 22, wherein:
the at least one further amino acid component is at most 2 weight percent relative to total weight of the composition.

24. The composition according to claim 16, further comprising:
vitamin B, wherein the vitamin B is selected from at least one of the following: Vitamin B6, vitamin B12, and folic acid.

25. The composition according to claim 24, wherein:
the vitamin B is between 0.05 weight percent and 0.5 weight percent relative to total weight of the composition.

26. The composition according to claim 24, wherein:
the vitamin B is between 0.05 weight percent and 0.2 weight percent relative to total weight of the composition.

27. The composition according to claim 16, further comprising:
a trace metal.

28. The composition according to claim 27, wherein:
the trace metal comprises zinc.

29. The composition according to claim 27, wherein:
the trace metal is between 0.05 weight percent and 0.2 weight percent relative to total weight of the composition.

30. The composition according to claim 16, for use in cell activation and/or induction of autophagy.

31. A nutritional supplement comprising or consisting of a composition according to claim 16.

32. The nutritional supplement according to claim 31, wherein:
the nutritional supplement comprises glucose, wherein the glucose is between 70 weight percent and 95 weight percent relative to total weight of the composition.

33. The nutritional supplement according to claim 32, further comprising:
a transport mediator.

34. The nutritional supplement according to claim 33, wherein:
the transport mediator comprises an agent activating glucose transporter GLUT1.

35. The nutritional supplement according to claim 33, wherein:
the transport mediator is between 0.5 weight percent and 10 weight percent relative to the total weight of glucose in the composition.

36. The nutritional supplement according to claim 33, wherein:
the transport mediator is selected from at least one of the following: phytopreparation, oestrogen-like natural substance, omega-3 fatty acid, and biguanide.
